(19) United States
(12) Patent Application Publication
(10) Pub. No.: US 2017/0290621 A1
(43) Pub. Date: Oct. 12, 2017

(54) Method for Cauterizing with Moxa Using a Moxibustion Device

(71) Applicant: Jeong Gil Jeong, Gyeongsangnam-do (KR)

(72) Inventor: Jeong Gil Jeong, Gyeongsangnam-do (KR)

(21) Appl. No.: 15/096,488

(22) Filed: Apr. 12, 2016

(51) Int. Cl. A61B 18/06

(52) U.S. Cl. CPC A61B 18/06; A61B 2018/00339

(57) ABSTRACT

The present invention relates to a cauterizing method for strengthening the circulation of gi • acupoints of a human body by using a moxibustion device.

### TECHNICAL FIELD

The present invention relates to a cauterizing method for strengthening a circulation of gi • acupoints of a human body by using a moxibustion device.

### BACKGROUND ART

In terms of Koreane Medicine, since a human body consists of a flow of “gi(vital energy)” and “acupoints”, when “gi” and “acupoints” are smoothly circulated, a health can be maintained, and when “gi” and “acupoints” causes a circulatory disturbance due to any cause, it is directly connected to an illness to exhibit responses in areas of a body or functions of organs.

A moxibution is a traditional therapy which infuses warm energy into meridians and acupuncture points located along a skin layer of the human body by cauterizing moxa cautery with moxa which is made from the dried Mug-wort (Artemisia argyi) to smoothly control the circulation of gi • acupoints and eventually to obtain an object for preventing and treating diseases.

Since meridians and acupuncture points are invisible, they are concepts which are not present in Western medical science.

Acupuncture point can be referred to as points (e.g., reaction point), where gi which is a material to moves the human body, i.e., energy is located on epidermis along a meridian line (e.g., reaction line) representing vessels of such gi and controls a flow of gi in the body, and can be used as points which treat diseases by using moxibustion, cupping, acupressure or acupuncture.

The total number of acupuncture points is about 1,000 adding up 365 main acupuncture points, newly-determined acupoints, and extra acupoints, etc. In acupuncture points, single acupoint include conception vessel and governor vessel, and double acupoint include twelve meridians.

When a moxa cautery is cauterized on the acupuncture points, the efficiency is each differently appeared according to the area having acupuncture point, and thus diseases are treated by each differently prescribing them according to the efficiency of acupoints. The efficiency of such moxa cautery has been widely known in treating diseases since a long time ago, but since the direct moxibustion is the cauterizing the moxibustion by directly burning the skin, it has a strong effect, but has disadvantages that a pain and scar can be occurred due to the burn.

A moxibustion device is the device designed so that a moxa cautery therapy is conveniently and effectively performed to obtain the effect without any direct contact with the skin. When a spot cauterized with moxa is set, if they are not moxibution expert, anyone can cauterize the moxa cautery by using the moxibustion device.

However, although the treatment to acupuncture points was practiced by the moxa cautery therapy with the moxibustion device, there were many cases that the complete recovery of disease and pain-relieving are not easily made.

### PRIOR TECHNICAL REFERENCES

### [Patent References]

U.S. Pat. No. 3,946,733

### DISCLOSURE

### Technical Problem

Therefore, inventor of the present application has been studied the cauterizing method for more strengthening a circulation of gi • acupoints by using the moxibustion device, and as a result, have been found that the circulation of gi • acupoints is strengthened by cauterizing with the moxibustion device in a serial combination manner and completed the present invention.

Accordingly, the object of the present invention is to provide the cauterizing method for strengthening a circulation of gi • acupoints in a human body by using the moxibustion device.

### Technical Solution

To accomplish the above-mentioned object, the present invention provides the cauterizing method for strengthening a circulation of gi • acupoints in a human body by using the moxibustion device.

### Advantageous Effects

The cauterizing method according to the present invention is useful in that the relief of the pain is easily felt and also, the effect is lasted for a long time, by cauterizing with existing moxibustion device which can be easily available on the market, in a serial combination manner.

### DESCRIPTION OF DRAWINGS

FIG. 1 is a drawing showing a front side of the main acupuncture points drawing of the human body.

FIG. 2 is a drawing showing a backside of the main acupuncture points drawing of the human body.

FIG. 3 is a drawing showing a moxibustion device.

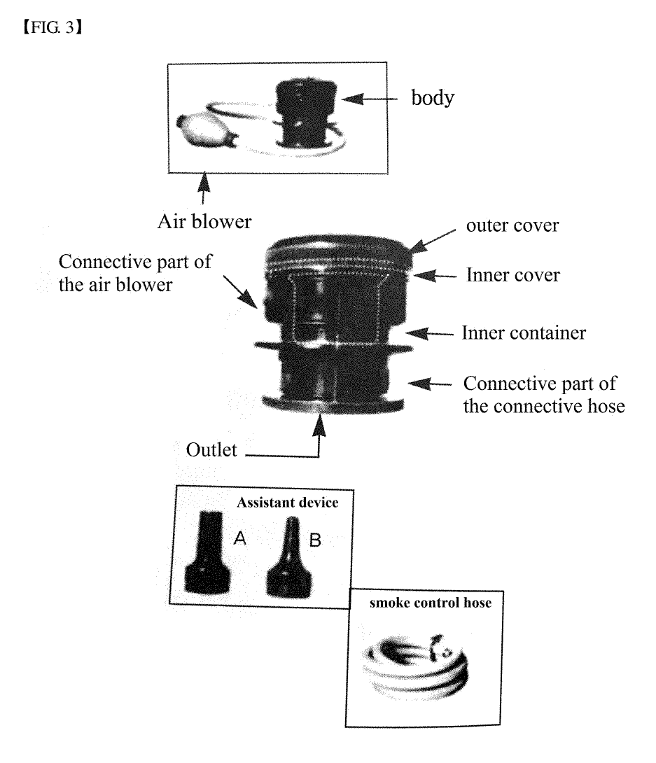

### BEST MODE FOR INVENTION

Hereinafter, the present invention will be explained in detail.

In the first embodiment of the present invention, the present invention provides a cauterizing method for strengthening a circulation of gi • acupoints in a human body by using the existing the moxibustion device.

The cauterizing method for strengthening a circulation of gi • acupoints in a human body by using a moxibustion device of the present invention comprises:

1) cauterizing on Taechu (No. 23), Left Pyesu (left of No. 25), Shinju (No. 35), Right Pyesu (right of No. 25), Left Kohwang (Left of No. 24), Left Shimsu (Left of No. 26) and the centre of both Shimsus (No. 26),

2) cauterizing on Right Shimsu (Right of No. 26), Right Kohwang (Right of No. 24), and from the centre of both Shimsus to the centre of both Kansus (No. 27) along the spine without any space,

3) cauterizing on Left Kansu (Left of No. 27), Right Kansu (Right of No. 27), and from the centre of both Kansus (No. 27) to the centre of both Pisus (No. 28) along the spine without any space,

4) cauterizing on Left Pisu (Left of No. 28), Right Pisu (Right of No. 28), and from the centre of both Pisus (No. 28) to Myungmun (No. 30) without any space,

5) continuously cauterizing on Left Sinsu (Left of No. 29), Right Sinsu (Right of No. 29), and from Myungmun (No. 30) to Yang-gwan (No. 31), Yeoshu (No. 32) and Jang-gang (No. 33),

6) turning the neck of the person to be cauterized from left to right three times,

7) continuously cauterizing on Cheondol (No. 13), Left Pyegeun (left of No. 12), Right Pyegeon (right of No. 12), Janjung (the intersection of the inter-nipple line and the anterior median line), from Janjung to the end of the solar plexus,

8) continuously cauterizing on Simgeul (No. 3), Left Bijung (left of No. 10), Right Bijung (right of No. 10), Wijung (No. 2), Jejung (No. 1), Kihae (No. 4), Danjeon (No. 6),

9) cauterizing on Left Chok-samri (left of No. 16), Right Chok-samri (right of No. 16), both Yongcheons (No. 19),

10) turning the neck of the person to be cauterized from left to right three times,

11) cauterizing on Left Myungsin (left of No. 22), the centre of both Myungsins (No. 22), Right Myungsin (right of No. 22),

12) turning the neck of the person to be cauterized from left to right three times, and

13) turning both ankles of the person to be cauterized outward three times under directly stretched state both ankles with relaxing them.

Any moxibustion device which can be commercially available can be used in the above cauterizing method.

The moxibustion device is basically consisted of a body and air blower, and it can further comprise an assistant device which is used to direct attach to an outlet such as a narrow part, an assistant device which is used on a part that the outlet part can not directly attach, such as an inside of the human body, and smoke control hose discharging smoke.

The basic method for using the above mixibustion device is well known in the art, but is preferably as follows:

1) Connecting the air blower to the body.

2) Opening the outer cover and then the inner cover.

3) Putting about 6 g of Mug-wort (small egg size, an amount of 25˜35 minutes of moxibustion) into the inner container and then lighting a fire on Mog-wort.

4) When the Mog-wort is sufficiently burned by the fire (1˜2 minutes), closing the inner cover and then the outer cover.

5) After cauterizing with pressing the air blower more fast, when an exhaust heat is hot, the cauterization is practiced with controlling heat temperature by pressing the air blower slowly.

In this case, any Mug-wort which can be commercially available can be used, and preferably middle grade Gangwha-mugwort (Artemisia princeps) of Korean is used.

Gangwha-mugwort comprises various physiologically active ingredients including flavonoid such as qupatilin, jaceosidin, etc., phenylpropanoid such as sesamin, etc. Gangwha-mugwort has an efficiency regarding the prevention and treatment for various diseases in terms of effective, and has an efficiency in inhibiting a hyperfunction such as odynolysis, sedation, determent, etc through the inhibitive action in terms of function.

The above cauterizing method is based on that the purified Mug-wort is put in the moxibustion device and the smoke of the Mug-wort is passed through acupunture points of the human body or pores around it to circulate gi.

Since the above cauterizing method uses the moxibustion device, when the moxibustion device is put on the parts which are estimated as being acupuncture points, a point of acupuncture points can be included in the circular border of the moxibustion device, and thus there is an advantage that the above cauterizing method can be easily practiced without professionalism that acupuncture points has to be exactly discovered.

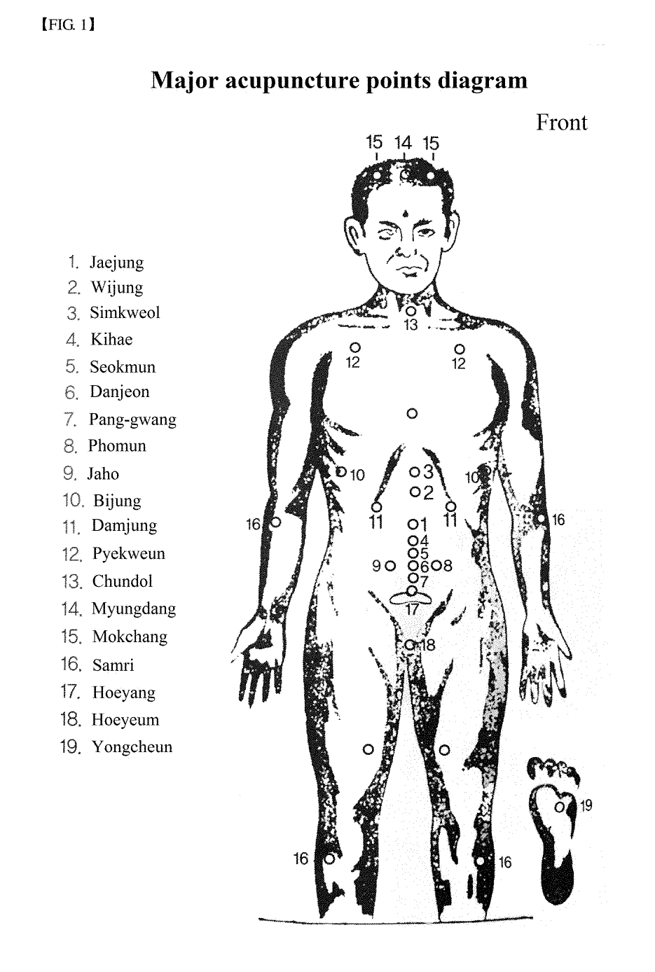

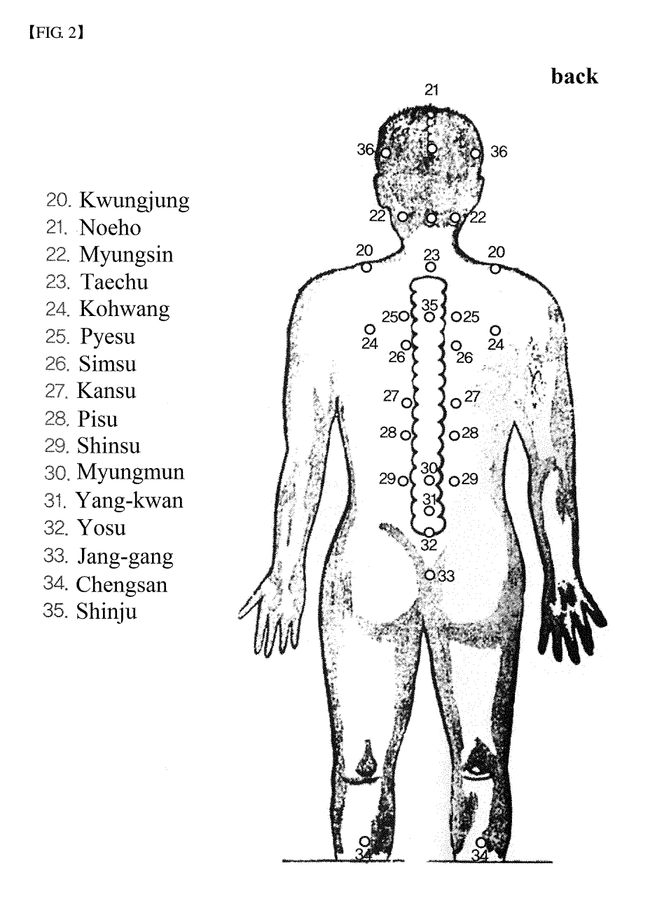

The above cauterizing method is characterized in cauterizing acupuncture points of Conception vessel (front of the human body, FIG. 1) and acupuncture points of Governor vessel (back of the human body, FIG. 2). In particular, the method is principally based on cauterizing from upward to downward, simultaneously with from left to right, along the order of the location of acupuncture points. Each acupuncture points of the human body as described in the above cauterizing method is depicted in FIGS. 1 and 2.

Preferably, acupuncture points to be cauterized according to the present invention are as follows:

Myungsin (No. 22): 1 chon superior to the superior border (the posterior hairline) of Taechu acupuncture point (No. 23), and 1.5 chon bilateral to the posterior median line

Taechu (No. 23): 1 chon superior to the first thoracic vertebra; bifurcation of the nerve

Shinju (No. 35): the concave part inferior to the third thoracic vertebra

Pyesu (No. 25): 1.5 chon bilateral to the inferior border of the third thoracic vertebra

Kohwang (No. 24): 3 chon bilateral to the inferior border of the fourth thoracic vertebra

Simsu (No. 26): 1.5 chon bilateral to the inferior border of the fifth thoracic vertebra

Kansu (No. 27): 1.5 chon bilateral to the inferior border of the ninth thoracic vertebra

Pisu (No. 28): 1.5 chon bilateral to the inferior border of the eleventh thoracic vertebra

Sinsu (No. 29): 1.5 chon bilateral to the inferior border of the second lumbar vertebra

Myungmun (No. 30): the median part inferior to the second lumbar vertebra

Yang-gwan (No. 31): 2 chon inferior to Myungmun

Yosu (No. 32): 3 chon inferior to Myungmun

Jang-gang (No. 33): 1 chon superior to anus

Chundol (No. 13): the central concave part in the anterior region of the neck

Pyegeun (No. 12): superior to the centre of the nipple, and concave part bilateral to the superior part of the third intercostal space

Bijung (No. 10): 3 chon inferior to the centre of the nipple, and 2 chon bilateral to the anterior median line

Junjung (Junjung): the intersection of the inter-nipple line and the anterior median line

Simkweol (No. 3): the central part between the end of the solar plexus and the centre of the umbilicus

Wijung (No. 2): the central part between the end of the solar plexus and the centre of the umbilicus

Jejung (No. 1): the centre of the umbilicus

Kihae (No. 4): 1.5 chon inferior to the centre of the umbilicus

Danjeon (No. 6): 3 chon inferior to the centre of the umbilicus

Chok-samri (No. 16): 3 chon inferior to the patella

Yongcheon (No. 19): the concave part antero-inferior to the sole; the junction between the great and the second toes, which is exhibited in a shape of Chinese Letter A when the toes are flexed

Wherein, the term ‘chon’ which is a basis for measuring acupuncture point locations is based on a finger joint of the cauterizing person.

The above cauterizing method is to locate the acupuncture point, which is out of position due to a usual wrong habitual behavior or overuse of the special parts of the body, to the regular position. That is, the above cauterizing method is to locate the vertebrae in position.

In the cauterizing method of the present invention, only the above acupuncture points are not acupuncture points for improving the circulation of gi • acupoints.

The cauterizing method of the present invention is preferably performed after allowing a person to meditate and breathe comfortably by crossing the legs in full or half lotus posture before practicing it. The full-lotus or half-lotus posture is to stand a waist straightly, thereby obtaining the desirable effect for strengthening the circulation of gi • acupoints according to the cauterizing method of the present invention.

In the above cauterizing method, when the cauterization is performed on acupuncture points of governor vessel, the half-lotus posture is preferable. When the cauterization is performed on acupuncture points of conception vessel, it is preferable that the half-lotus posture, or the lying posture with taking the posture that heels of the feet pull outwards and the toe parts draw forward to the body under the situation that a non-high pillow is used and both soles of feet are extended straightly with drawing a chin forward to the body, is held.

When cauterizing on Chok-samri (No. 16) acupuncture point in 9) of the cauterizing method, it is preferable that the cauterizing is performed in the posture of the corresponding leg is stood.

When cauterizing on Yongcheon (No. 19) acupuncture point in 9) and 14) of the cauterizing method, it is preferable to take the posture wherein heels of feet pull outwards and the toe parts draw forward to the body under the situation that both feet are extended straightly.

The above cauterizing method takes a total of 40 minutes to 45 minutes, and is preferable to be performed within 2 hours a day.

Preferably, at first the cauterization is performed over 30 seconds to 1 minute per one acupuncture point. When the heating power of the fire of Mug-wort is stronger, the cauterization is performed within 30 seconds, preferably over 10 to 20 seconds. When the heating power of the fire of Mug-wort is stronger, the cauterization is performed over 1 to 3 seconds per one acupuncture point. If the fire of Mug-wort is remained after completing all of the cauterizing methods, above steps can be repeated from the first.

Each cauterizing is usually performed until feeling hot for each of acupuncture points and meridian, and in the case of the first cauterizing, 30 seconds to 1 minute per acupuncture points is proper since the hot feeling is not felt immediately.

When a pain such as tingling sensation on feet etc. is felt during the cauterizing method of the present invention, full-lotus or half-lotus posture is held again by changing both legs. Nevertheless, if the pain is not relived, the pain can be relieved by cauterizing on Yoncheon (No. 19) acupuncture point or Jang-gang (No. 33) acupuncture point of tingled feet.

During the cauterization, the person to be cauterized should pay attention not to raise his (her) chin above or not to twist his (her) body so as to effectively make the circulation of gi • acupoints.

It is preferable that drinking or intaking of meats (chicken, pork, duck meat, etc.) is abstained, and non-smoking and abstinence are made. In addition, it should take notice that the cauterizing method of the present invention is not to be performed on a patient with cancer or a pregnant woman.

The condition that the circulation of gi • acupoints is strengthened means that the verterbrate of the human body is returned in position with extending it straightly, and the above main acupuncture points and meridian are restored the original right circulation route.

Such condition that the circulation of gi • acupoints is strengthened can be noticed from feelings that pores of the skin is evidently wide-open, sweat comes out in hands and feet, the whole body is warm, the skin is moist, eyes light up, the head is clear, an appetite becomes good, sweat comes out under the arm, and the like.

Preferably, the condition that the circulation of gi • acupoints is strengthened can be identified by the visual check for that a material with a elastic round shape in collagen form is protruded in the posterior hairline of Taechu acupuncture point (No. 23)

The condition that the circulation of gi • acupoints is strengthened could be varied depending on the bodily constitution, but it could be identified within at least 1˜3 days after performing the cauterizing method of the present invention, and its effect was lasted for a long time (several days˜dozens of days).

The cauterizing method of the present invention is for relieving pain, preferably backache and all sorts of pain, and for alleviating hypertension.

Hereinafter, the present invention will be explained in more detail through Working Examples. However, these Examples are provided to illustratively explain the present invention and the scope of the present invention is not limited into the examples.

<A Complete Recovery of Lumbar Dislocation and Sciatic Neuralgia>

As a result of that the cauterizing method of the present invention was practiced on a patient with the dislocation of the second lumbar vertebra and sciatic neuralgia, it was identified that the lumbar dislocation and sciatic neuralgia were completely recovered.

<Alleviation of Hypertension>

As a result of that the cauterizing method of the present invention was practiced with a frequency of 3˜4 times on a patient with hypertension, it was be identified that a normal blood pressure in the degree of 126˜127 was maintained without taking any hypertension drug.

<A Treatment of the Symptom that the Nerve of Both Legs is Trapped>

As a result of that the cauterizing method of the present invention was practiced for about a month and a half on a patient with the symptom that both legs are tingling and weakening, it was identified that such symptoms did not appear.

<Alleviation of Symptom of Cervical Disc>

As a result of that the cauterizing method of the present invention was practiced on a patient with the severe symptom of cervical disc, it was identified that the pains of hands, arms and shoulder were almost alleviated, except for the feeling that hands and arms are tingling a little.

The person with an ordinary skill in the art to which the present invention subjects can understand that the present invention can be embodied into the modified form within the scope that the present invention is not departed from the essential feature. Therefore, the disclosed Examples should be considered as not the limited view, but the explanatory view. The scope of the present invention is appeared not in the above-mentioned description, but in the appended claims, and all differences within the equivalent range should be considered as being included in the present invention.

1. A cauterizing method using a moxibustion device, which comprises:
1) cauterizing on Taechu (No. 23), Left Pyesu (left of No. 25), Shinju (No. 35), Right Pyesu (right of No. 25), Left Kohwang (Left of No. 24), Left Shimsu (Left of No. 26) and the centre of both Shimsus (No. 26),
2) cauterizing on Right Shimsu (Right of No. 26), Right Kohwang (Right of No. 24), and from the centre of both Shimsus to the centre of both Kansus (No. 27) along the spine without any space,
3) cauterizing on Left Kansu (Left of No. 27), Right Kansu (Right of No. 27), and from the centre of both Kansus (No. 27) to the centre of both Pisus (No. 28) along the spine without any space,
4) cauterizing on Left Pisu (Left of No. 28), Right Pisu (Right of No. 28), and from the centre of both Pisus (No. 28) to Myungmun (No. 30) without any space,
5) continuously cauterizing on Left Sinsu (Left of No. 29), Right Sinsu (Right of No. 29), and from Myungmun (No. 30) to Yang-gwan (No. 31), Yeoshu (No. 32) and Jang-gang (No. 33),
6) turning the neck of the person to be cauterized from left to right three times,
7) continuously cauterizing on Cheondol (No. 13), Left Pyegeun (left of No. 12), Right Pyegeon (right of No. 12), Janjung (the intersection of the inter-nipple line and the anterior median line), from Janjung to the end of the solar plexus,
8) continuously cauterizing on Simgeul (No. 3), Left Bijung (left of No. 10), Right Bijung (right of No. 10), Wijung (No. 2), Jejung (No. 1), Kihae (No. 4), Danjeon (No. 6),
9) cauterizing on Left Chok-samri (left of No. 16), Right Chok-samri (right of No. 16), both Yongcheons (No. 19),
10) turning the neck of the person to be cauterized from left to right three times,
11) cauterizing on Left Myungsin (left of No. 22), the centre of both Myungsins (No. 22), Right Myungsin (right of No. 22),
12) turning the neck of the person to be cauterized from left to right three times, and
13) turning both ankles of the person to be cauterized outward three times under directly stretched state both ankles with relaxing them.

2. The cauterizing method according to claim 1, wherein a circulation of gi • acupoints in a human body is strengthened.

3. The cauterizing method according to claim 1, wherein the person holds a full-lotus or half-lotus posture before practicing the method.

4. The cauterizing method according to claim 1, wherein the person holds the half-lotus posture when the cauterization is performed on acupuncture points of Governor vessel.

5. The cauterizing method according to claim 1, wherein the person holds the half-lotus posture, or the lying posture with taking the posture that heels of the feet pull outwards and the toe parts draw forward to the body under the situation that a non-high pillow is used and both soles of feet are extended straightly with drawing a chin forward to the body, when the cauterization is performed on acupuncture points of Conception vessel.

6. The cauterizing method according to claim 1, wherein the cauterization is performed within 2 hours.

7. The cauterizing method according to claim 1, wherein at first the cauterization is performed over 30 seconds to 1 minute, and when the heating power of the fire of Mug-wort is stronger, the cauterization is performed over 10 to 20 seconds, and when the fire of Mug-wort is stronger, the cauterization is performed over 1 to 3 seconds, per one acupuncture point.

8. The cauterizing method according to claim 1, characterized in that it is for alleviating pains.
